(19) United States
(12) Patent Application Publication
(10) Pub. No.: US 2007/0053522 A1
(43) Pub. Date: Mar. 8, 2007

(54) Method and apparatus for directional enhancement of speech elements in noisy environments

(76) Inventors: Daniel James Murray, Kitchener (CA); Gary Allan Young, Kitchener (CA)

Correspondence Address: Gowling Lafleur Henderson LLP;1 First Canadian Place, 100 King Street West, Toronto, ON, M5X 1G5, CA

(21) Appl. No.: 11/220,605

(22) Filed: Sep. 8, 2005

(51) Int. Cl. A61F 11/06

(52) U.S. Cl. 381/71.6

(57) ABSTRACT

A listening device and respective method for processing speech audio signals present in noisy acoustical sound waves captured from an adjacent environment for persons with normal hearing. The device comprises a housing for providing acoustical and mechanical coupling to a user's ear, the housing having a first portion for positioning in the ear and an elongated second portion extending from the first portion. The device also comprises a pair of spaced apart microphones positioned on a line-of-sight reference vector and supported by the housing, at least one of the microphones located in the elongated second portion of the housing, the microphones configured for capturing the acoustical sound waves from the environment including speech related elements and non-speech related elements. A digital signal processor is supported by the housing and is configured for digitally processing the captured acoustical sound waves to identify and select the speech related elements propagating towards the second portion in the vicinity of along the line of sight vector and for enhancing the signal strength of the selected speech related elements with respect to other of the elements in the captured acoustical sound waves to generate a processed acoustical digital signal. A receiver located in the first portion is used for converting the processed acoustical digital signals into processed analog acoustical signals and for transmitting the processed analog acoustical signals into the user's ear.

This invention relates generally to the digital processing of speech contained in acquired sound waves in noisy environments by a personal listening device.

### BACKGROUND OF THE INVENTION

Environments typically have a number of competing sounds that disrupt conversation between two or more individuals. Examples of these environments include restaurants, pubs, trade shows, sports venues and other social situations in which conversational speech is partially masked by undesirable competing speech and other background noise. This type of interfering noise typically masks important speech information and can impede conversation occurring between people with otherwise normal hearing. Although prior art in current hearing aids, for example, do provide noise reduction functionality, there is a disadvantage in that they are not appropriate for persons with normal hearing since they are configured for hearing loss compensation, calibrated on a person-by-person basis based on individual hearing loss characteristics, therefore may not be suitable for use in enhancing conversational speech from the disrupting background noise inherent in social environments, for persons with normal hearing.

It is an object of the present invention to provide a listening system and method to obviate or mitigate at least some of the above presented disadvantages.

### SUMMARY OF THE INVENTION

Current hearing aids have a disadvantage in that they are configured for persons with hearing loss to provide hearing loss compensation, calibrated on a person-by-person basis based on individual hearing loss characteristics. Therefore, hearing aids are not suitable for use in enhancing conversational speech from the disrupting background noise inherent in social environments, for persons with normal hearing. Contrary to current hearing aids, which compensate for hearing loss, there is provided a listening device and respective method which focuses exclusively on capturing speech in the presence of background noise, without providing any specific compensation for hearing loss, by processing speech audio signals present in noisy acoustical sound waves captured from an adjacent environment. The device comprises a housing for providing acoustical and mechanical coupling to a user's ear, the housing having a first portion for positioning in the ear and an elongated second portion extending from the first portion. The device also comprises a pair of spaced apart microphones positioned on a line-of-sight reference vector and supported by the housing, at least one of the microphones located in the elongated second portion of the housing, the microphones configured for capturing the acoustical sound waves from the environment including speech related elements and non-speech related elements. A digital signal processor is supported by the housing and is configured for digitally processing the captured acoustical sound waves to identify and select the speech related elements propagating towards the second portion in the vicinity of along the line of sight vector and for enhancing the signal strength of the selected speech related elements with respect to other of the elements in the captured acoustical sound waves to generate a processed acoustical digital signal. A receiver located in the first portion is used for converting the processed acoustical digital signals into processed analog acoustical signals and for transmitting the processed analog acoustical signals into the user's ear.

One aspect provided is a listening device for processing speech audio signals present in acoustical sound waves captured from an adjacent environment, the device comprising: a housing for providing acoustical and mechanical coupling to a user's ear, the housing having a first portion for positioning in the ear and an elongated second portion extending from the first portion; a pair of spaced apart microphones positioned on a line-of-sight reference vector and supported by the housing, at least one of the microphones located in the elongated second portion of the housing, the microphones configured for capturing the acoustical sound waves from the environment including speech related elements from a first source and non-speech related elements from a second source; a digital signal processor supported by the housing and configured for digitally processing the captured acoustical sound waves to identify and select the speech related elements propagating towards the second portion in the vicinity of along the line of sight vector and for enhancing the signal strength of the selected speech related elements over that of the non-speech related elements in the captured acoustical sound waves to generate a processed acoustical digital signal; and a receiver located in the first portion for converting the processed acoustical digital signals into processed analog acoustical signals and for transmitting the processed analog acoustical signals into the user's ear.

A second aspect provided is a method for processing speech audio signals present in acoustical sound waves captured from an adjacent environment, the method comprising the steps of: capturing the acoustical sound waves from the environment including speech related elements from a first source and non-speech related elements from a second source by a pair of spaced apart microphones positioned on a line-of-sight reference vector, at least one of the microphones located in an elongated portion of a device housing positioned adjacent to a user's ear; digitally processing the captured acoustical sound waves to identify and select the speech related elements propagating towards the second portion in the vicinity of along the line of sight vector; enhancing the signal strength of the selected speech related elements over that of the non-speech related elements in the captured acoustical sound waves to generate a processed acoustical digital signal; converting the processed acoustical digital signals into processed analog acoustical signals; and transmitting the processed analog acoustical signals into the user's ear.

### BRIEF DESCRIPTION OF THE DRAWINGS

Exemplary embodiments of the invention will now be described in conjunction with the following drawings, by way of example only, in which:

FIG. 1A shows a top view of a listening device;

FIG. 1B shows a side view of the device of FIG. 1A;

FIG. 1C, shows a bottom view of the device of FIG. 1A;

FIG. 2 is a block diagram a digital signal processor of the device of FIG. 1A;

FIG. 3 shows a frequency response graph of the digital signal processor of FIG. 2;

FIG. 4 shows a block diagram of a processing algorithm of the digital signal processor of FIG. 2; and

FIG. 5 is an example operation of the device of FIG. 2.

### DETAILED DESCRIPTION OF THE PREFERRED EMBODIMENTS

Listening Device 10 Components

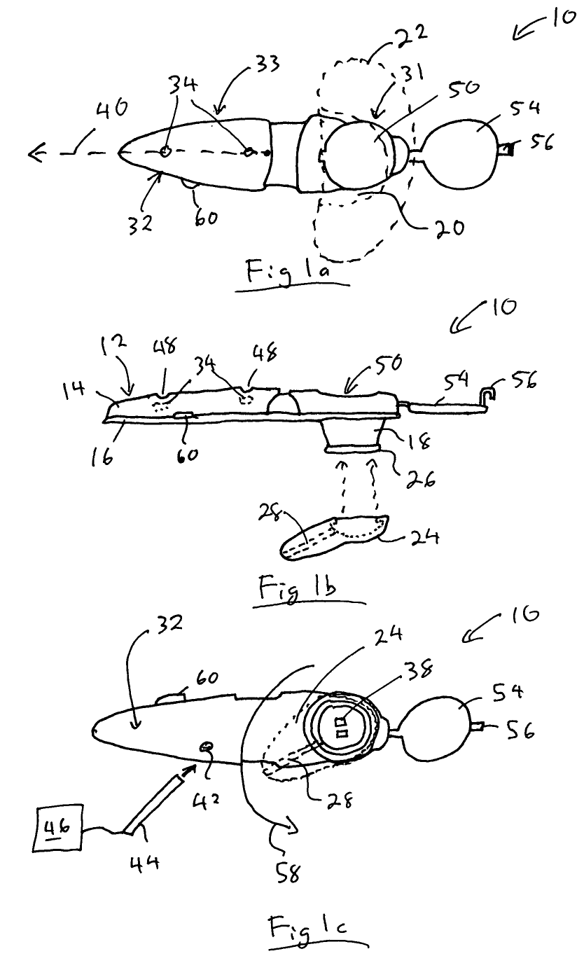

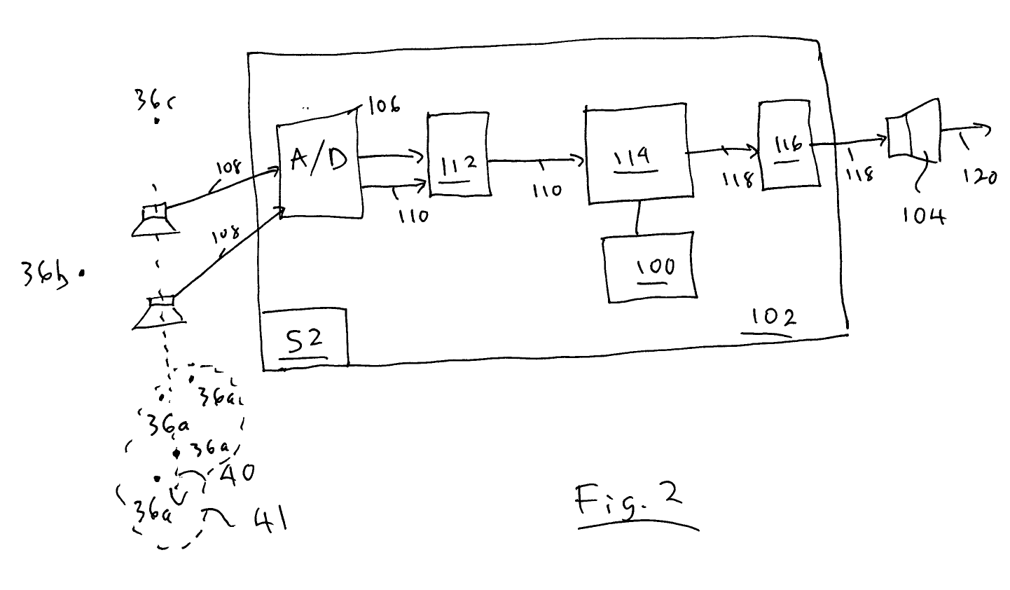

Referring to FIGS. 1a, 1b, 1c, a personal listening device 10 has a housing 12 consisting of a top shell 14 and a bottom shell 16, made from such as but not limited to ABS plastic. The housing 12 has a main portion 31, for accommodating a battery compartment 50 and an ear port 18, coupled to an extended portion 33, for accommodating the location of one or more spaced apart microphones 34. The device 10 uses two or more spaced apart microphones 34, for example both located in the extended portion 33, for capturing of sound waves emanating from multiple sources 36a,b,c in the user's local environment 38. The device 10 uses directional, noise reduction, and feedback compensation signal processing (directed by an algorithm 100—see FIG. 2), of sound waves captured by the spaced apart microphones 34, to improve the clarity and quality of desired speech audio signals mixed with undesired background noise (e.g. desired speech sound waves captured from source 36a along with undesired background noise captured from sources 36b and 36c).

The device 10 acts to enhance the sound quality of desired speech audio signals (e.g. emanating from source 36a) by facing the device 10 (i.e. line-of-sight 40) to the source 36a of the sounds, thereby using the directional sound reduction processing techniques of the algorithm 100 to filter out in real-time the undesired noise coming from other directions (e.g. from behind and beside the user—from sources 36b and 36c). The algorithm 100 of the device 10 processes digitized signals of the captured sound waves for the purpose of noise reduction for speech fricatives/elements included in the sound waves. It is recognized that processing to compensate for individual hearing impairment (i.e. varying insensitivity to selected frequency ranges—e.g. hard of hearing for high frequencies versus adequate hearing for low frequencies), as is known in the art, is preferably not accommodated in the algorithm 100 as part of the directional processing. Accordingly, the device 10 is designed for helping to enhance the quality of speech/conversations in noisy environments 38 for users with normal hearing capabilities.

The device 10 can be configured to increase the ability of a device user with normal hearing to enhance the user's ability to hear speech in noisy environments 38. The targeted typical noise environment can be such as but not limited to a noisy restaurant, meeting, or other social setting. The signal gain of the device 10 (e.g. supplied by a digital signal processor 102—See FIG. 2) can be limited to levels required to replace the ear canal's natural resonant peak that can be lost with the insertion of an ear tip 24 and can help to provide sufficient functionality of the directional algorithm 100. A maximum power output of processed sound waves 120 (see FIG. 2) of the device 10 are preferably limited to sound levels that are below the maximum safe output level guidelines for normal ear 22 hearing, e.g. 104 dB SPL in a 2 cc coupler. It is recognized that normal hearing can be defined as hearing capability that does not have an appreciable level of hearing impairment (due to accident, age-related, genetic, etc. . . . ), as determined by a medical hearing specialist.

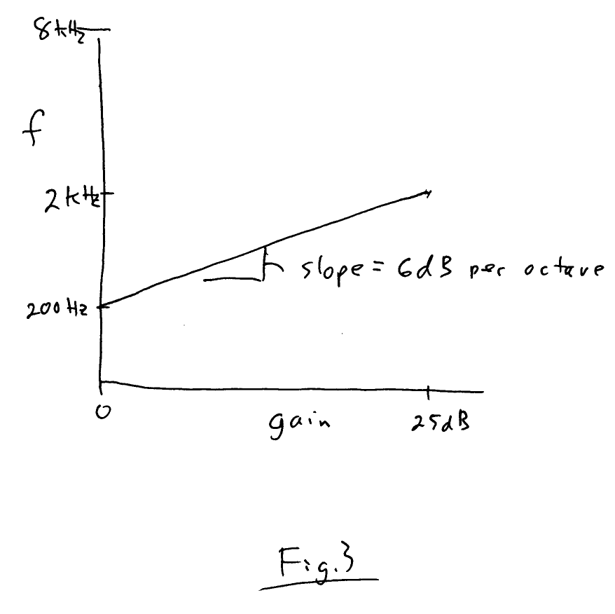

Referring again to FIGS. 1a, 1b, 1c, the bottom shell 16 of the housing 12 has the ear port 18 that extends into a concha bowl 20 of a user's ear 22. There are 4 sizes of ear tip 24, that can be releasably secured to a lip 26 of the ear port 18 using for example a snap fit. Examples of the ear tip 24 can be such as those used in Blue Tooth™ headsets, as is known in the art. The ear tip 24 provides acoustical and mechanical coupling of the listening device 10 to the ear concha bowl 20, preferably without full occlusion and minimal feedback, and is preferably made of a resilient plastic material for adapting to the shape of the user's concha bowl 20. The ear tip 24 has a vent 26 to help prevent occlusion and an acoustical channel 28 for directing processed sound waves 120 from a receiver 30 of the device 10 to the user's ear canal/ear drum (not shown). It is recognized that the ear tip 24 can rotate about the ear port 18 (indicated by arrow 58), thus providing for selection of the desired line-of-sight 40 by the user when the device 10 is coupled to the user's ear 22. Further, a calibration/programming port 42 can be used during manufacturing of the device 10 for inserting of a probe 44 connected to a calibration program 46. The calibration program 46 is used to calibrate the processing algorithm 100 to enhance the sound quality of speech elements captured in the sound waves from the sources 36a,b,c according to a selected frequency response 200 (see FIG. 3), and directional processing parameters according to the actual manufactured spacing of the microphones 34 and to compensate for any differences in sensitivity between the microphones 34 of the device 10. It is recognized that the programming port 42 can be a factory programming port 42 not accessible to the end user of the device 10. Further, the port 42 can be in locations other than as shown, for example accessible through a battery compartment 50.

Referring again to FIGS. 1a, 1b, 1c, the top shell 14 has two openings 48 for providing acoustic access of the sound waves from the sources 36a,b,c to the spaced apart microphones 34 housed in the interior of the housing 12. The housing 12 design is visible and mainly situated external to the ear 22, such that the extended portion 33 (e.g. an elongated dagger shaped extension) extends from the ear port 18 and is designed to house the two microphones 34, in order to provide a desirable visual form factor of the device 10 with optimized microphone 34 separation for directional processing of speech fricatives/elements contained in the sound waves, as captured from the source(s) 36a (located along the line-of-sight 40 defined by the microphone 34 spacing). It is recognized that one of the microphones 34 could be positioned in the extended portion 33 while the other microphone could be located in the main/base portion 31, such that the optimal line-of-sight 40 and spacing of the microphones 34 is maintained, as further discussed below. The top shell 14 also accommodates a battery compartment 50 for housing a battery 52 to supply operational power to the device 10. A compartment cover 54 is hinged at one end with a locking mechanism 56 at the other end for releasably securing the cover to the top shell 14, thus retaining the battery 52 within the compartment 50. The battery cover 54 is hinged to facilitate battery 52 replacement as needed.

In general, the housing 12 interior is configured to house the device electronics (see FIG. 2), namely: an AMIS Toccata Plus DSP chipset 102 (or other digital signal processor as desired); the two (or more) matched microphones 34; one receiver 104; one battery 52; and a volume control 60 with a built-in on-off switch. The housing 12 can be symmetrical by design so that it can be worn on either ear 22 thereby minimizing the need for the user to make adjustments for left and right ear usage.

Speech in Sound Waves

In general, continuous speech is a set of complicated audio signals. Speech signals are usually considered as voiced or unvoiced, but in some cases they are something between these two. Voiced sounds consist of fundamental frequency (F0) and its harmonic components produced by vocal cords (vocal folds). The vocal tract can modifies this excitation signal causing formant (pole) and sometimes anti-formant (zero) frequencies. Each formant frequency has also an amplitude and bandwidth. Speech can contain sound waves representing such as but not limited to: Vowels; Diphthongs; Semivowels; Fricatives; Nasals; Plosives; and Affricates. For example, speech fricatives are those sounds which have a noise-like quality and are generated by forcing air from the lungs through a tight constriction in the vocal tract, such as the ‘s’ in sea or ‘th’ in thread. With purely unvoiced sounds, there is no fundamental frequency in excitation signal and therefore no harmonic structure either and the excitation can be considered as white noise. The airflow is forced through a vocal tract constriction which can occur in several places between glottis and mouth. Some sounds are produced with complete stoppage of airflow followed by a sudden release, producing an impulsive turbulent excitation often followed by a more protracted turbulent excitation. Unvoiced sounds are also usually more silent and less steady than voiced ones. Whispering is the special case of speech, such that when whispering a voiced sound there is no fundamental frequency in the excitation and the first formant frequencies produced by vocal tract are perceived.

It is recognized by example, that speech signals can have the fundamental frequency of about 100 Hz and the formant frequencies with vowel /a/ can be approximately 600 Hz, 1000 Hz, and 2500 Hz respectively, with vowel /i/ the first three formants can be 200 Hz, 2300 Hz, and 3000 Hz, and with /u/ 300 Hz, 600 Hz, and 2300 Hz. In general, speech elements of sound waves can be found in the frequency range of approximately 100 Hz to 8 KHz, for example. The signal processor 102 and associated algorithm 100 are configured to recognize speech elements in the sound waves emanating from the sources 36a,b,c and to decrease the amplitude of all sound waves other than those of speech contained in the sound waves from the source(s) 36a located along the line-of-sight 40 (in front of the device 10 in a vicinity region 41 associated as part of the line-of-sight 40). The processing of the captured sound waves can be done to filter out undesired sounds using frequency modulation, amplitude modulation, and delay-sum directional techniques possible when two microphone signals are available, or a combination thereof.

For example, referring to FIG. 2, the signal processor 102 and associated algorithm 100 would enhance speech elements present in the captured sound waves from the source 36a, reduce the presence of non-speech sound waves captured from source 36a, and reduce the presence of all sound waves captured from the sources 36b,c located off the line-of-sight 40 (e.g. to the side and/or rear of the device 10). Further, it is recognized that the signal processor 102 and associated algorithm 100 could also identify the speech elements contained in the sources 36b,c and decrease their presence in the processed sound waves 120, while enhancing the speech contained in the sound waves captured from the source 36a. This enhancement of speech in the processed sound waves 120 from the desired source 36a while decreasing the presence (e.g. amplitude) of speech in the processed sound waves 120 from the undesired source(s) 36b,c could be done as a priority while effectively preserving the presence of non-speech related sounds present in the sound waves captured from one or more of the sources 36a,b,c. This preferential treatment of speech related sound waves from the desired source(s) 36a could be selected by the user of the device 10 depending upon the environment 38 noise characteristics, i.e. select for enhancement of speech only or select for the enhancement of speech with the simultaneous decrease of diminishment of the non-speech related sounds. It is recognized that the device 10 may not eliminate the undesired sounds from the captured sound waves, rather the device 10 may just reduce them in amplitude relative to the desired sounds.

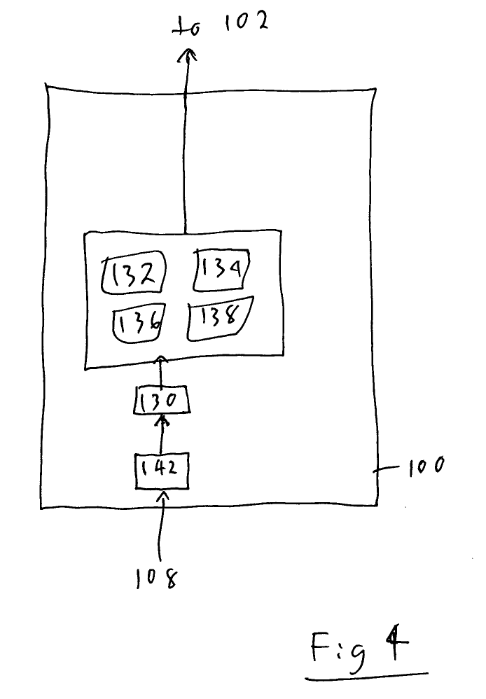

A further operational example would be use of the device 10 in either restaurant/bar social settings or when walking or driving or operating heavy machinery, e.g. in open air external environments 38. A selection module 130 (see FIG. 4) could be used to select between local and outdoor environments 38, where for local environments 38 the device 10 operation would be optimized for isolation of desired speech from undesired speech including noise reduction, while in an outdoor environment 38 or other larger environment setting the processing of the processor 102 would allow for speech optimization only while allowing background noise present in the captured sound waves to remain substantially uncompensated in the processed sound waves 120. A further embodiment would have background noise uncompensated in local environments 38 while compensated in more open environments 38, as selected by the user of the device 10 by the selection module 130 (see FIG. 4). Further, it is recognised that the venting of the ear tip 24 can prevent total occlusion, so the user of the device 10 can hear loud sounds from behind or beside.

Digital Signal Processing

Referring to FIG. 2, the device 10 has five basic parts, namely: the housing 12 designed for providing microphone 34 spacing, housing the device electronics, and for providing functional acoustical and mechanical coupling to the user's ear 22; the spaced apart microphones 34 for picking up the sound waves from the sources 36a,b,c and sending the analog sound waves as electrical signals to the digital signal processor 102; the digital signal processor 102 for digitally processing the captured sound waves according to the associated processing algorithm 100—operation of which is further described below by way of example; the receiver 104 for converting electrical signals received from the signal processor into acoustic signals and directing the processed acoustic signals into the ear 22 canal; and the battery 52 for supplying operational electrical power to requisite device 12 components.

Referring again to FIG. 2, the signal processor 102 of the device 10 takes sound waves captured from the sources 36a,b,c, which undergo analog-to-digital conversion, digital processing, and then transformation back into sound by digital-to-analog conversion. The digital processing of the captured sound waves is preferably done in real time or with a negligible user perceptible delay (e.g. less than 10 milliseconds) so that the user does not notice a discrepancy between sound perception and the visual aspects of speech. The signal processor 102 has an input port 106 for receiving electrical signals 108 from the spaced apart microphones 34 and for converting the electrical signals 108 to digital signals 110. The digital signals 110, representing essentially unprocessed sound information of the sound waves captured from the sources 36a,b,c, can be stored in a FIFO input memory buffer 112 prior to processing by a processor 114 (in conjunction with the programmed operations of the processing algorithm 100). The signals 110, once processed, can be output to a FIFO output memory buffer 116 as processed signals 118 before being sent to the receiver 104 for conversion back into analog acoustical sound waves 120 that are directed into the ear 22 of the device 10 user. It is recognized that the processed sound waves 120 differ by a predetermined time difference threshold from the original captured sound waves of the sources 36a,b,c by noise reduction and directional processing techniques implemented by the processor 114 and associated algorithm 100.

Referring again to FIGS. 1 and 2, the device 10 is a battery-powered, ear-worn directional audio device that improves the clarity and quality of desired speech related sounds (from sources 36a) in the presence undesired background noise (from sources 36b,c). The background noise can include both speech and non-speech related sound waves. The user of the device 10 can focus on desired speech related sounds by facing the source 36a of those sounds and the device 10 will use the digital directional processing technology of the processor 102 and associated algorithm 100 to filter out undesired sounds coming from the other directions (e.g. from behind and beside the user).

The spaced apart microphones 34 are positioned in the extended portion 33, for example, both along the line-of-sight 40 such that the signal processor 102 can use sound delay, as is known in the art, of the same sound waves captured by each of the microphones 34 to minimize distracting noise from the same sound waves originating from sources 36b located towards the rear of the device 10 (i.e. approximately 180 degrees referenced from the line-of-sight 40 of the extended portion 33) and to minimize distracting noise from the same sound waves originating from sources 36c located more towards the side of the device 10 (i.e. approximately 90/270 degrees referenced from the line-of-sight 40 of the extended portion 33), while emphasizing the desired same sound waves emanating from the source 36a located generally in-front of the device (i.e. approximately 0 degrees referenced from the line-of-sight 40 of the extended portion 33). Accordingly, the digital processor 102 and associated algorithm 100 are configured to preferably filter out unwanted sound waves captured from sources 36b,c located to the sides and rear of the extended portion 33 (e.g. in an arc from approximately after 0 degrees to just before 360 degrees), while enhancing those desired sound waves captured from source(s) 36a located generally in-front of the extended portion 33 in the vicinity of along the line-of-sight reference vector 40. The line-of-sight vector 40 is positionable by the user of the device 10 so as to preferably point in the same direction as the user's face or line of sight. It is recognized that the above-stated angle magnitudes/directions are given as an example only and as such the signal processing operation of the device 10 can give preferential processing treatment to same sound waves received from sources 36a in the general vicinity of in-front of the extended portion 33 along the line-of-sight 40. In general, signal 108 attenuation is done for those signals 108 determined to originate from sources 36b,c located approximately in the range of −90 degrees to +270 degrees from the line-of-sight 40 vector. It is recognized that the location range of the preferred sources 36a would be in a vicinity region 41 associated as part of the line-of-sight 40). For example, all captured sound waves determined to have a time difference (when compared) below a certain predetermined difference threshold would be considered as part of the vicinity region 41 and therefore become identified as coming from preferred sources 36a (e.g. those speech related elements from the preferred sources 36a would be enhanced over other audio elements present in the captured sound waves—i.e. those non-preferred elements would be determined to be from non-preferred sources 36b,c).

Referring to FIG. 3, the device 10 is designed having a non-programmable, fixed frequency response profile 200, such that the elements (e.g. fricatives) of speech present in the captured audio signals 108 (see FIG. 2) are amplified by a set or otherwise predefined optimal gain “curve” (e.g. to 25 dB gain at 2 kHz), used by the algorithm 100 to help isolate the speech sounds from the background noise of the sound waves captured from any of the sources 36a,b,c. As an example, the profile 200 can be represented as 6 dB per octave rising slope, starting at 200 Hz, rising to a peak gain of 20 to 25 dB at 2 kHz. and then the gain falls off to about 0 dB gain at 7500 Hz to 8000 Hz. It is recognized that the device 10 has two microphones 34, by example, that have sufficient separation (e.g. 14 mm) to provide optimum directionality processing for amplitude/frequency enhancement of speech elements in the captured sound waves, i.e. the microphone spacing is configured for beam optimization for frequencies approximately in the 100/200 Hz to 7000/8000 Hz range. The device 10 sits in the ear such that both microphones 34 align along user positioned line-of-sight 40 in order to achieve targeted directionality of the signal processing.

Directional Processing Algorithm 100

Referring to FIG. 4, the signal processing algorithm 100 is used to direct the digital signal processing of the processor 114. The algorithm 100 has a number of modules 128 for providing a specified level of noise reduction in the captured signals 108, in combination with good sound quality and feedback cancellation, wherein the “noise reduction” can be characterized by example in the reduction of undesired speech elements and non-speech elements captured from varying directions with respect to the line-of-sight 40 defined by the two or more spaced apart microphones 34. The algorithm 100 can be used to remove obvious relatively constant noise such as fan hum and loud transients such as clanging dishes. The device 10 should work well in reverberant as well as non-reverberant rooms, however it is recognized that the algorithm 100 may not completely eliminate the undesired background noise, where certain background noises may not be attenuated at all depending on the reverberant nature of the environment 38 and the nature of the noise. However, in general the algorithm 100 will process the signals 108 to reduce the level of undesired background noise (e.g. speech elements and/or non-speech related sound) originating from behind/beside the device 10 relative to target sounds (e.g. speech related elements) arriving from the front of the user, enabling the user to better hear most target sounds from the front (i.e. in the vicinity of along the line-of-sight 40).

The following modules 128, or a selection thereof, can be activated within the algorithm 100, such as but not limited:

Directional Processing Module 132

The module 132 uses 2-microphone 34 (for example) directional processing for providing the noise reduction for the undesired sounds present in the captured sound waves from the environment 38 of the device 10. The directional processing of the module 132 uses the profile 200 (see FIG. 3) to amplify speech related sounds arriving from the front of the listener while attenuating sounds (speech and/or non-speech related sounds) arriving from the sides/rear of the device 10. For example, sounds arriving from 180 degrees with respect to the line-of-sight 40 can be attenuated by 10 dB. It is noted that the spacing of the microphones 34 can be matched to parameters such as but not limited to: the frequency range of the desired speech related elements in the captured signals 108 (e.g. 100-8000 Hz); the sound capturing capabilities of the microphones 34; and/or the processing capabilities of the digital signal processor 102. The module 132 uses directional technology that by comparing the signals 108 captured by each of the microphones 34, the module 132 can detect the direction (with respect to the line-of-sight 40) from which the captured sound waves arrives according to comparison to a time difference threshold, i.e. the location of the respective source 36a,b,c in the environment 38 either in or outside of the preferred vicinity 41. One method for direction determination is using the slight time differences between the compared sound waves that occur due to the finite speed of sound traveling to each of the spaced apart microphones 34.

Noise Reduction Module 134

The noise reduction module 134 of the signal processing algorithm 100 is aimed at improving overall sound quality of the desired signals enhanced in the processed sound waves 120.

Output Compression Module 136

The output compression module is used to limit the output level (i.e. dBs) of the processed sound waves 102 to determined safe levels and to help reduce receiver 104 distortion due to excessive signal 118 strength.

Feedback Cancellation Module 138

The feedback cancellation module 138 helps to reduce feedback introduced into the signals 108.

End of Battery Life Tone Module 140

This module 140 will generate a recognizable tone to inform the user of the device 10 that the battery 50 is near the end of its useful life and should be changed.

Filter Mode Module 130

This module 130 is used for selection of which filtering mode the algorithm 100 should operate, e.g. filter out only speech related elements from the signals 108 or filter out both speech related elements and non-speech related elements from the signals 108. The module 130 can also be used to give a selected angular range (or other measurement—e.g. quadrant of the region outside of the vicinity region 41) for assigning sources 36a,b,c in the respective selected region(s) of the environment 38 to user preferred signal processing. For example, captured sound waves from sources 36c located in the region of the rear of the device 10 could be processed to remove both speech and non-speech related audio signals while captured sound waves from sources 36b located in the region of beside the device 10 (considered part of the vicinity region 41) could be processed to remove only non-speech related sound waves. In this example, the user of the device 10 would be able to interact in conversations with multiple people positioned in-front and to the side (e.g. peripherally) with respect to the user (and line-of-sight 40), such that only non-speech related audio signals would be attenuated for those audio signals emanating from in-front and to the side of the user, while both speech and non-related speech audio signals emanating from behind the user would be attenuated (e.g. speech and other sounds). This example of selective filtering based on direction with respect to the line-of-sight 40 would help the user focus on the conversation between the user and the group of people position in-front and to the side, while helping the user to ignore any sound distractions from the rear. Accordingly, the user could use the module 130 though a selection button (not shown) to adjust the size and scope of the vicinity region 41. Further, it is recognized that there could be more than one level of vicinity region 41, as desired, for example two vicinity regions with varying degrees of attenuation and filter modes. It is recognized that the module 130 could also be used to adjust a level of attenuation of the undesired audio signals, as well as a ratio of attenuation between speech and non-speech related audio signals, e.g. attenuate speech related signals by 5 dB and non-speech related signals by 10 dB.

Characterization Module 142

This module 142 is used to determine from the signals 108 which of the signals 108 represents speech related sounds and which of the signals represents non-speech related sounds. For example, one method of determination would be to analyze which sounds occur in a selected speech frequency range and/or which of the sounds contains speech characterizations (e.g. fundamental frequencies, harmonics, and other identifiable elements such as but not limited to Vowels; Diphthongs; Semivowels; Fricatives; Nasals; Plosives; and Affricates as is known in the art. The determination of speech versus non-speech related sounds could be used by the filter module 130 during filtering of the signals 108.

Operation of the Device 10

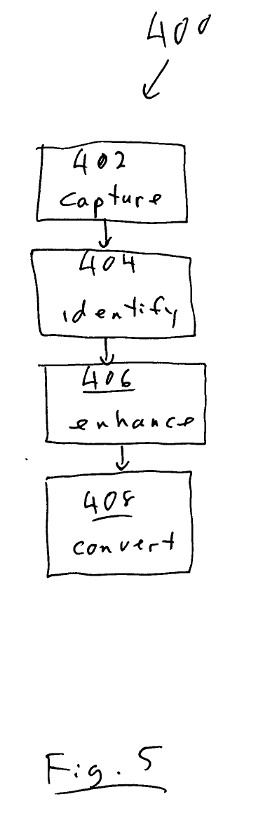

Referring to FIGS. 2 and 5, an example operation 400 of the device 10 is shown for processing speech audio signals present in acoustical sound waves captured from an adjacent environment. The first step 402 is to capture the acoustical sound waves from the environment 38 including speech related elements and non-speech related elements by the pair of spaced apart microphones 34 positioned on the line-of-sight 40 reference vector. The next step 404 is to digitally process the captured acoustical sound waves 108 by the digital processor 102 to identify and select the speech related elements propagating towards the device in the vicinity of along the line of sight vector 40, as performed by the module 130. The next step 406 is to enhance the signal strength of the selected speech related elements with respect to other of the elements in the captured acoustical sound waves to generate a processed acoustical digital signal 118. The enhancement of the signal is done by the processor 114 in conjunction with the algorithm 100. The next step 408 is to convert the processed acoustical digital signals 118 by the receiver 104 into processed analog acoustical signals 120 and to transmit the processed analog acoustical signals 120 into the user's ear 22.

It is recognized that the algorithm 100 and the digital signal processor 102 are implemented on a computing device as art of the listening device 10. Further, it is recognized that the algorithm 100 and digital signal processor 102 could be configured other than as described, for example a configuration such as but not limited to a combined digital signal processor including an integrated algorithm. Further, it is recognized that the functional components of the digital signal processor 102 and the algorithm 100 could be represented as software, hardware, or a combination thereof.

We claim:

1. A listening device for processing speech audio signals present in acoustical sound waves captured from an adjacent environment, the device comprising:
a housing for providing acoustical and mechanical coupling to a user's ear, the housing having a first portion for positioning in the ear and an elongated second portion extending from the first portion;
a pair of spaced apart microphones positioned on a line-of-sight reference vector and supported by the housing, at least one of the microphones located in the elongated second portion of the housing, the microphones configured for capturing the acoustical sound waves from the environment including speech related elements from a first source and non-speech related elements from a second source;
a digital signal processor supported by the housing and for configured for digitally processing the captured acoustical sound waves to identify and select the speech related elements propagating towards the second portion in the vicinity of along the line of sight vector and for enhancing the signal strength of the selected speech related elements over that of the non-speech related elements in the captured acoustical sound waves to generate a processed acoustical digital signal;
a receiver located in the first portion for converting the processed acoustical digital signals into processed analog acoustical signals and for transmitting the processed analog acoustical signals into the user's ear.

2. The device of claim 1 further comprising an ear tip configured for coupling to the first portion for providing user adjustable alignment of the line-of-sight reference vector to give targeted directionality of the digital signal processor.

3. The device of claim 1 further comprising a fixed frequency response profile for use by the digital signal processor for amplifying speech related elements while attenuating non-speech related elements.

4. The device of claim 3, wherein the fixed frequency response profile includes a 6 dB per octave rising slope rising to a peak gain of 20 to 25 dB at 2 kHz.

5. The device of claim 3, wherein the digital signal processor processes the captured acoustical sound waves using a technique selected from the group comprising: frequency modulation; amplitude modulation; and delay-sum directional techniques.

6. The device of claim 3, wherein the microphone spacing of the spaced apart microphones is based on a parameter selected from the group comprising: a frequency range of the desired speech related elements in the captured acoustical sound waves; sound capturing capabilities of the microphones; and processing capabilities of the digital signal processor.

7. The device of claim 6, wherein the microphone spacing is configured for beam optimization for frequencies approximately in the 100 Hz to 8000 Hz frequency range.

8. The device of claim 7, wherein the microphone spacing is 14 mm.

9. The device of claim 3 further comprising a selection module coupled to the digital signal processor for selecting a first region in the adjacent environment with respect to the line-of-sight reference vector, the region including the first source producing the speech related elements.

10. The device of claim 9 further comprising the selection module for selecting a second region in the adjacent environment with respect to the line-of-sight reference vector, the second region including the second source producing the non-speech related elements.

11. The device of claim 10 further comprising a filter module for applying a first filter mode to the first region and a second filter mode different from the first filter mode to the second region.

12. The device of claim 9, wherein the first region is selected by a setting selected from the group comprising: an angular range and a quadrant of the adjacent environment.

13. The device of claim 11, wherein the first filter mode reduces non-speech related elements captured from the first region.

14. The device of claim 13, wherein the second filter mode reduces both speech and non-speech related elements captured from the second region.

15. The device of claim 14, wherein the second filter mode attenuates the speech related elements by 5 dB and the non-speech related elements by 10 dB.

16. A method for processing speech audio signals present in acoustical sound waves captured from an adjacent environment, the method comprising the steps of:
capturing the acoustical sound waves from the environment including speech related elements from a first source and non-speech related elements from a second source by a pair of spaced apart microphones positioned on a line-of-sight reference Vector, at least one of the microphones located in an elongated portion of a device housing positioned adjacent to a user's ear;
digitally processing the captured acoustical sound waves to identify and select the speech related elements propagating towards the second portion in the vicinity of along the line of sight vector
enhancing the signal strength of the selected speech related elements over that of the non-speech related elements in the captured acoustical sound waves to generate a processed acoustical digital signal;
converting the processed acoustical digital signals into processed analog acoustical signals; and
transmitting the processed analog acoustical signals into the user's ear.

17. The method of claim 16 further comprising the step of applying a fixed frequency response profile by the digital signal processor for amplifying speech related elements while attenuating non-speech related elements.

18. The method of claim 16 further comprising the step of selecting a first region in the adjacent environment with respect to the line-of-sight reference vector, the region including the first source producing the speech related elements.

19. The method of claim 18 further comprising the step of selecting a second region in the adjacent environment with respect to the line-of-sight reference vector, the second region including the second source producing the non-speech related elements.

20. The method of claim 19 further comprising the step of applying a first filter mode to the first region and a second filter mode different from the first filter mode to the second region.
